(19) United States
(12) Patent Application Publication
(10) Pub. No.: US 2012/0053676 A1
(43) Pub. Date: Mar. 1, 2012

(54) Implantable Prosthetic Vascular Valves

(76) Inventors: David N. Ku, Decatur, GA (US); Prem Midha, Rolla, MO (US); Harris Bergman, Marietta, GA (US)

(21) Appl. No.: 13/319,137

(22) PCT Filed: May 7, 2010
(86) PCT No.: PCT/US10/34093

§ 371 Date: Nov. 7, 2011

Related U.S. Application Data
(60) Provisional application No. 61/176,285, filed on May 7, 2009.

(51) Int. Cl. A61F 2/82; A61F 2/24

(52) U.S. Cl. 623/1.26; 623/2.17

(57) ABSTRACT

In one embodiment, an implantable prosthetic valve includes a body that defines an inlet end and an inlet orifice, the body being constructed of a flexible biocompatible material, an outlet that extends from the body and that defines an outlet end and an outlet orifice, the outlet being constructed of a flexible biocompatible material, and an inner passage that extends through the body and the outlet from the inlet orifice to the outlet orifice to enable fluid to flow through the valve, wherein the outlet orifice is open when the valve is in its natural unloaded state and is closed when fluid pressure is applied to the outlet from a position downstream of the outlet.

### FIELD OF THE DISCLOSURE

This present disclosure is directed to implantable prosthetic venous valves designed to replace diseased, damaged, or clinically incompetent valves in the human venous system. It is recommended for, but not limited to, implantation in the iliac, femoral, or saphenous veins in humans.

### BACKGROUND

The human venous system from the lower extremities contains a number of one-way valves that function in allowing forward (antegrade) blood flow to the right atrium of the heart while preventing reverse (retrograde) flow to the feet. Using the muscle action of the calf, or the “peripheral heart,” the body is able to overcome gravitational forces to maintain blood flow back to the heart. The valves thus prevent blood from pooling in the lower extremities. Physiologically functioning valves are capable of withstanding very high proximal pressure gradients with minimal leakage, and can open at very low distal pressure gradients. However, for many patients, venous function is severely compromised by chronic venous disease (CVD), caused by chronic venous insufficiency (CVI).

CVI affects nearly one million new patients every year, and causes health problems such as varicose veins, ulceration, swelling, and, in more severe cases, deep vein thrombosis and pulmonary embolism. Venous reflux causes 80 to 90 percent of CVI and is the result of incompetent venous valves. The most common type of incompetence, secondary incompetence, often results in complete destruction of the valve leaflets. Venous reflux due to secondary incompetence is rarely surgically repaired, and when it is, the repair seldom lasts. When secondary incompetence occurs in the deep venous system, valve replacement is the only viable treatment.

There are two main options in deep venous valve replacement: 1) transplantation or transposition and 2) prosthetic implantation. The first vein valve autotransplant in a human patient in was performed in 1982. However, even after more than 20 years of refinement, venous transplant surgery is still used only in few cases, only after medication, physical rest and therapy, and other less invasive surgical procedures have been tried or considered. Valve transplant or transposition can cause unnecessary trauma to the patient's leg, and most procedures require indefinite post-operative anti-coagulation treatment. Problems may also arise even prior to surgery; for instance, it can be difficult to find a suitable donor valve. This is evidenced by the fact that 30 to 40 percent of auxiliary vein valves, which are often used for superficial femoral venous valve replacement, are found to be incompetent prior to harvesting. The challenges of using a native vein valve for transplantation or transposition thus increase the need for a suitable prosthetic vein valve. Unfortunately, there has yet to be a prosthetic venous valve developed that has demonstrated the necessary functional performance for operating satisfactorily in human physiologic conditions. While various designs have been pursued in the past, many such designs possess shortcomings that prevent them from being a sufficiently functional design.

### BRIEF DESCRIPTION OF THE FIGURES

The disclosed valves can be better understood with reference to the following drawings. The components in the drawings are not necessarily to scale, emphasis instead being placed upon clearly illustrating the principles of the present disclosure. In the drawings, like reference numerals designate corresponding parts throughout the several views.

FIG. 1 is a top perspective view of an implantable valve.

FIG. 2 is a cross-sectional view of the valve of FIG. 1 taken along line A-A.

FIG. 3 is a cross-sectional view of the valve of FIG. 1 taken along line B-B.

FIG. 4 is a side view of the valve of FIG. 1.

FIG. 5 is a front view of the valve of FIG. 1.

FIG. 6 is a top perspective view of the valve of FIG. 1, shown with an outlet of the valve in a closed position.

FIG. 7 is a side view of a second embodiment of an implantable valve.

FIG. 8 is a front view of the valve of FIG. 7.

FIG. 9 is a side view of a third embodiment of an implantable valve.

FIG. 10 is a front view of the valve of FIG. 9.

FIG. 11 is a top perspective view of a fourth embodiment of an implantable valve.

FIG. 12 is a top perspective view of a fifth embodiment of an implantable valve.

FIG. 13 is a top view of a sixth embodiment of an implantable valve.

FIG. 14 is a top view of a seventh embodiment of an implantable valve.

FIG. 15A-15E are schematic views illustrating an embodiment of a method of implanting of a prosthetic valve within a vein.

### DETAILED DESCRIPTION

The present disclosure generally relates to prosthetic valves and methods of use and manufacture thereof. The valves of the present disclosure can be used in non-biological systems, but are generally designed as implantable, prosthetic valves for use in the human vascular system, particularly the venous system. The valves are particularly suited for use in the venous system because they allow antegrade blood flow towards the heart when subjected to a very low distal pressure gradient (e.g., that caused by contraction of leg muscles), and also prevent retrograde blood flow and leakage when subjected to physiologically high proximal pressure. In some embodiments, the valves are biocompatible, flexible, have low thrombogenicity, and are sufficiently durable to withstand multiple cycles of opening and closing in physiologic conditions.

The valves comprise a generally cylindrical tube having an inlet, a body, and an outlet. In some embodiments, the outlet comprises at least two leaflets. The outlet is in the open position in a relaxed state and therefore the valves may be referred to as “normally open” or “naturally open” valves. In the open position, the outlet enables fluid to flow through the valve in the forward direction from the inlet to the outlet. The outlet closes, however, upon the application of pressure from the direction of the outlet to reduce or prevent backflow/reflux of fluid back through the valve from the outlet to the inlet. The naturally open design of the valves provides a low amount of flow resistance allowing the blood to move freely in the antegrade direction.

In some embodiments, the valves are made of a flexible material to further enhance performance of the valves. The valves may be designed to have varying flexibility in the different valve components. For instance, in some embodiments the stiffness of the inlet and body is greater than that of the outlet and leaflets. The flexibility of the valve leaflets creates larger openings for flow and reduces the occurrence of high shear stress regions. In some embodiments, the valves contain no material that touches the venous wall in the location downstream of the base of the valve leaflets. By specifically having no mural material downstream of the base of the leaflets, the opening area of the outlet is large and stagnant blood clotting is reduced.

The valves of the present disclosure can be implanted in a patient using various procedures, including, but not limited to, a minimally invasive catheter procedure or more conventional surgical procedures (e.g., a venotomy), and can be affixed using various methods including, but not limited to, sutures, a stent or stent system, and hooked or barbed protrusions. The values further can be implanted using endoscopic insertion and fixation techniques.

For a prosthetic implantable vein valve to be optimally functional, the valve should typically have the following features. The valve should be able to open and allow antegrade (forward) flow with little resistance. The valve should also withstand physiologic proximal pressures of about 100 mm Hg or greater, while preventing reflux (reverse flow) and keeping leakage less than about 5 mL/second. The valve should have low thrombogenicity, should cause minimal pain to the patient, and should have the durability to last at least about 500,000 cycles. The valve should not become obstructed after implantation, lest it block blood flow. The valve should have low thrombogenicity with no platelet attachment under high shear after 1 hour of perfusion with whole blood. Prosthetic valves should also preferably meet the demands of vein distensibility, as the vein diameter expands from about 1.4 to about 2.0 times the normal vein diameter when subjected to pressures of only about 50 mm Hg. Ideally, this means the prosthetic valve should flexibly conform to the curves and bends of veins.

In general, the present disclosure describes implantable prosthetic valves designed to meet the functional criteria, set forth above, of a valve placed in the venous system. The valves are designed to allow substantially unrestricted antegrade flow (e.g., allows antegrade flow under a pressure gradient of about 5 mm Hg or less) and to minimize reflux and leakage, and are also easy to manufacture. The valves are designed to be biocompatible, have low thrombogenicity, and can also be used in other body vessels, particularly those that conduct primarily unidirectional flow. The valves can also be used in non-biological systems.

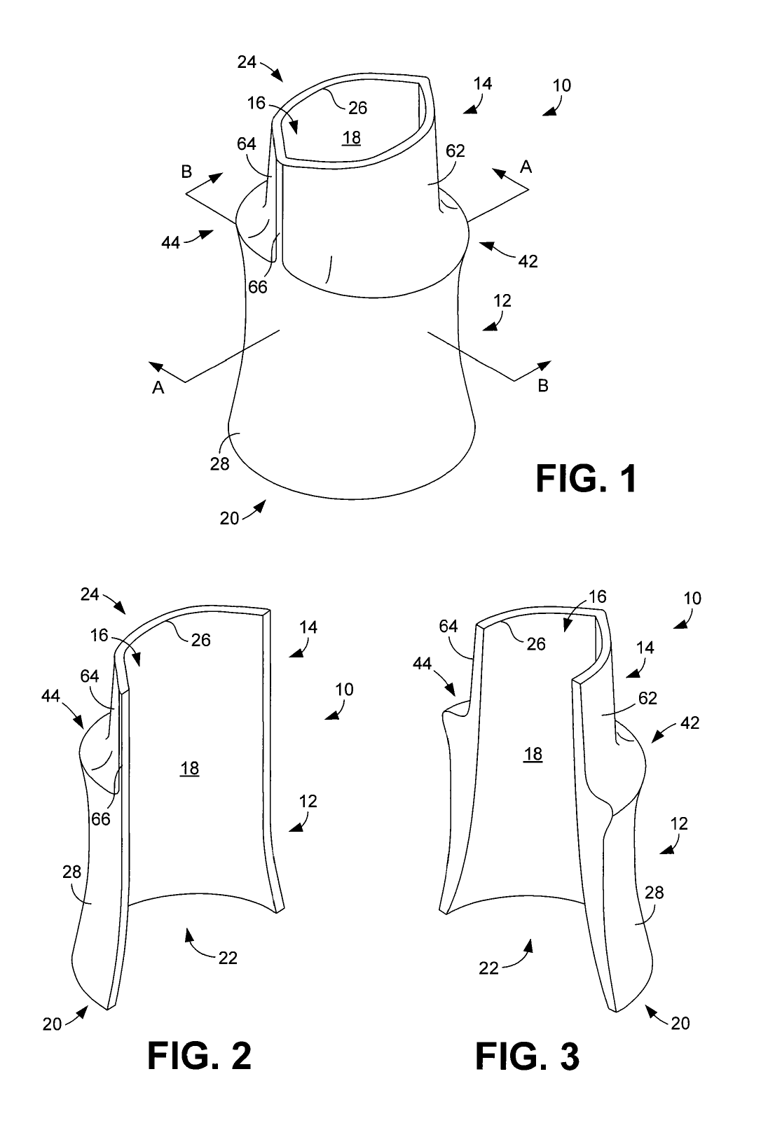

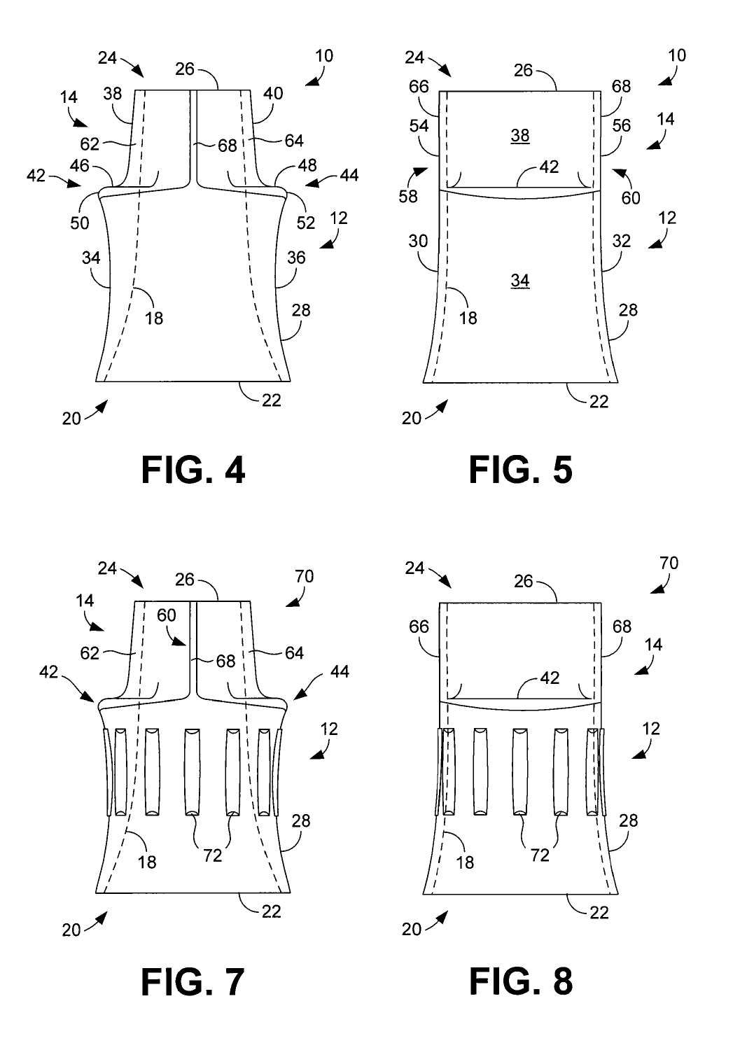

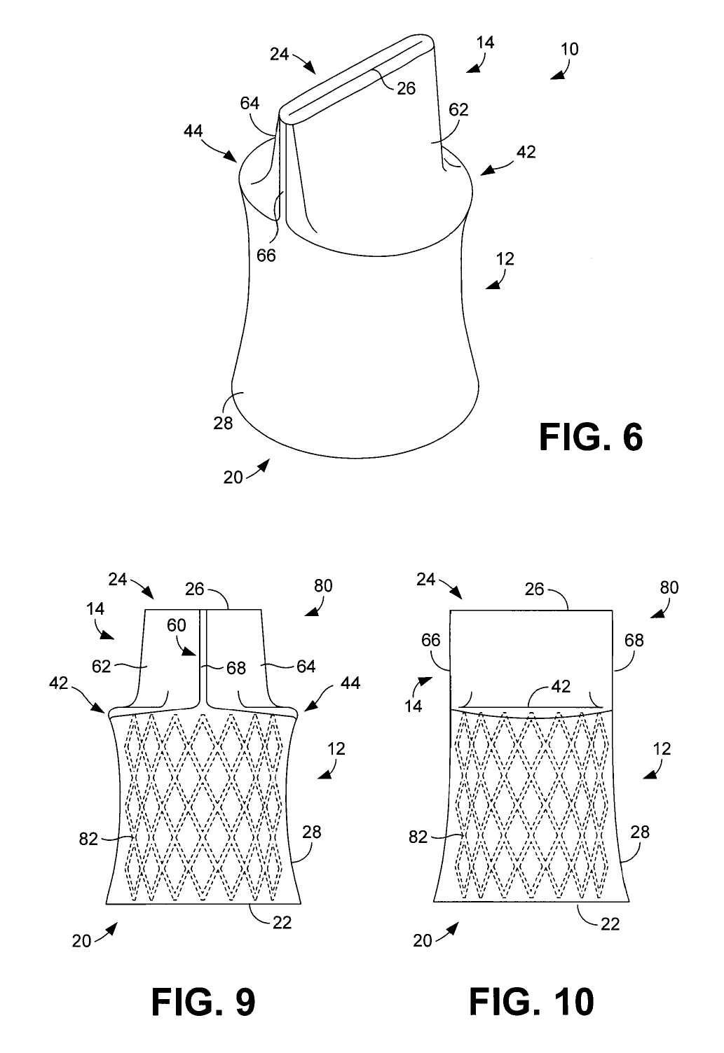

FIGS. 1-6 illustrate a first embodiment of an example implantable valve 10. In FIGS. 1-5, the valve 10 is shown in its natural, open state. In FIG. 6, the valve 10 is shown in its closed state. As is apparent from the figures, the valve 10 comprises a generally cylindrical body 12 from which extends a generally cylindrical outlet 14. Together, the body 12 and the outlet 14 form an inner passage 16 through which fluid, such as blood, can flow. The inner passage 16 is defined by inner walls 18 of the body 12 and the outlet 14. In the illustrated embodiment, the inner walls 18 form continuous, smooth surfaces. The body 12 forms an inlet end 20 of the valve 10 that comprises an inlet orifice 22 through which fluid can enter the inner passage 16. The outlet 14 forms an outlet end 24 of the valve 10 that comprises an outlet orifice 26 through which fluid can exit the passage 16. As is indicated in FIGS. 4 and 5, the inner walls 18 of the passage 16 curve inwardly from the inlet orifice 22 to the outlet orifice 26 such that the cross-sectional area of the passage decreases as the passage is traversed from the inlet end 20 of the valve 10 to its outlet end 24. In some embodiments, the curvature of the inner walls 18 decreases from the inlet end 20 to the outlet end 24 such that the walls are nearly parallel with the longitudinal or axial (flow) direction of the valve 10 near the outlet end.

As is most clearly apparent in FIGS. 4 and 5, the outer surfaces 28 of the body 12 are curved. In the illustrated embodiment, the outer surfaces 28, like the walls 18 of the inner passage 16, curve inwardly from the inlet end 20 of the body 12. However, the curvature of the body 12 varies about its periphery. As is shown in the front view of FIG. 5, the side edges 30 and 32 of the body 12 curve inwardly from the inlet end 20 to the outlet 14. As the side edges 30, 32 are traversed from the inlet end 20 to the outlet 14, their curvature decreases such that the side edges are nearly parallel to each other adjacent the point at which the body 12 ends and the outlet 14 begins, such that the side edges are generally parabolic in shape. However, as is shown in the side view of FIG. 4, the front and rear edges 34 and 36 of the body 12 curve inwardly from the inlet end 20, become nearly parallel with each other near the center of the body, and curve outwardly from the center of the body to the point at which the body ends and the outlet 14 begins, such that the front and rear edges are generally hyperbolic in shape.

Generally speaking, the outlet 14 is smaller in cross-section than the body 12. As is shown in FIG. 4, the width of the outlet 14, as measured from the front side 38 to the rear side 40 of the outlet, is smaller that the width of the body 12, as measured from its front side 34 to its rear side 36. Because of that, opposed ledges 42 and 44 are formed on the front and rear of the valve 10, respectively, at the junction between the body 12 and the outlet 14. The ledges 42, 44 protrude outwardly from the valve 10 and therefore may be referred to as protrusions. The ledges 42, 44 comprise generally planar top surfaces 46 and 48 that are substantially perpendicular to the longitudinal or axial (flow) direction of the valve 10. The ledges 42, 44 also comprise curved outer edges 50, 52 that mark the transition from the body 12 to the outlet 14. As can be appreciated when FIGS. 1-6 are considered together, each ledge 42, 44 is substantially crescent shaped (when viewed from above) with its widest extent near the middle of the ledge and tapering toward each opposed end. As is also shown in FIG. 4, the front and rear sides 38, 40 angle inwardly toward each other from the ledges 42, 44 to the outlet end 24 such that the outlet 14 has a frustoconical shape.

With reference to FIG. 5, the width of outlet 14, as measured from a first side 54 to a second side 56 of the outlet, is substantially the same as the width of the body 12, as measured from its first side 30 to its second side 32 near the junction between the outlet and the body.

Referring next to FIG. 1, the outlet 14 and the outlet orifice 26 has a generally elliptical cross-section. More particularly, the outlet 14 and the outlet orifice 26 have a generally lemon-shaped cross-section that is in part due to opposed seams 58 and 60 at which opposed leaflets 62 and 64 of the outlet are joined. It is noted that term “seam” is not intended to imply that the leaflets 62, 64 are separately formed components that are later connected together, although such fabrication is possible. The seams 58, 60 include planar edge surfaces 66 and 68 that are generally parallel with the longitudinal or axial (flow) direction (see FIG. 5) and that are coplanar with the outer surfaces 28 of the body 12.

The leaflets 62, 64 are thin walled so that they can collapse together when reverse fluid flow occurs, as with retrograde blood flow. FIG. 6 illustrates the leaflets 62, 64 when the valve 12 and its outlet 14 is in the closed position. In some embodiments, such closure can be effected by reverse flow pressures as low as 5 mm Hg. In other words, pressure downstream of the outlet 14 causes the leaflets 62, 64 to close.

As described in greater detail below, the valve 10 can be unitarily formed of a single piece of material, such as a flexible, biocompatible polymeric material. In some embodiments, the valve 10 can be formed using a suitable molding process. In some embodiments, the seams 58, 60 of the outlet 14 can be reinforced with additional and/or stiffer material to increase durability and prevent fatigue.

FIGS. 7 and 8 illustrate a second embodiment of an implantable valve 70. The valve 70 is similar in many ways to the valve 10. Therefore, like components have been identified with like numerals from FIGS. 1-6. Unlike the valve 10, however, the valve 70 includes multiple longitudinal or axial ribs 72 that are provided on the outer surfaces 28 of the body 12 of the valve. The ribs 72 are aligned with the flow direction of the valve 70 and are spaced equal distances from each other around the periphery of the body 12. In use, the ribs 72 provide structural rigidity to the body 12, and therefore the valve 70.

FIGS. 9 and 10 illustrate a third embodiment of an implantable valve 80. The valve 80 is also similar in many ways to the valve 10. Therefore, like components have been identified with like numerals from FIGS. 1-6. Unlike the valve 10, however, the valve 80 includes an integral stent 82, which is shown in an expanded state. In some embodiments, the stent 82 comprises a self-expanding metallic stent (SEMS). In some embodiments, the stent 82 is embedded within the body 12 of the valve 80, for example by placing the stent in a mold that is used to form the valve.

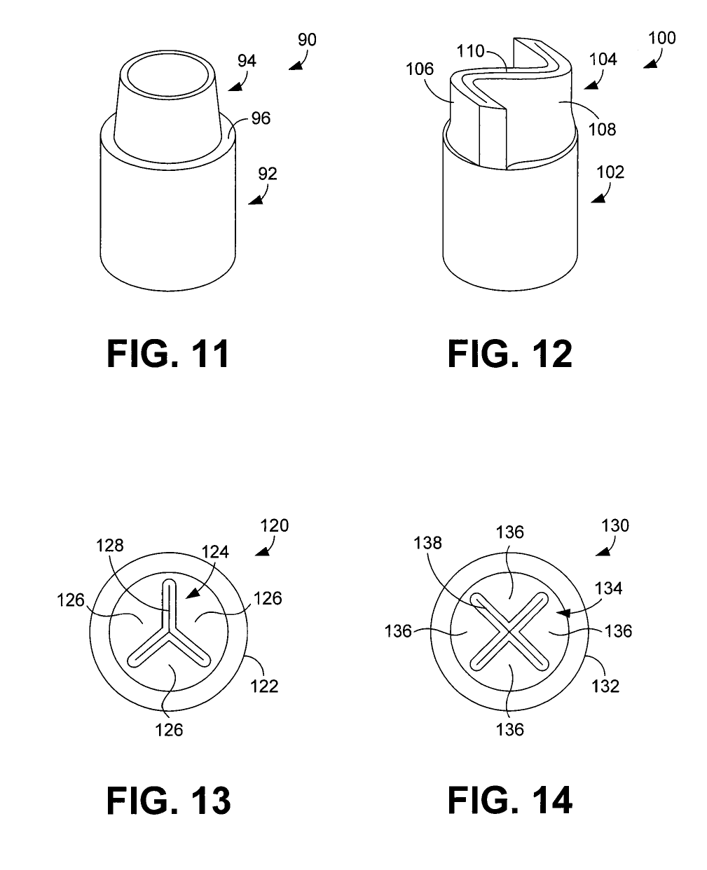

FIG. 11 illustrates a fourth embodiment of an implantable valve 90. The valve 90 is similar to the valve 10, except that the body 92 is generally cylindrical and the outlet 94 that extends from the body is generally frustoconical. Because the outlet 94 is generally frustoconical, it does not comprise distinct leaflets as do the previously-described valves. The difference in size between the body 92 and the outlet 94 results in the formation of an endless ledge 96.

FIG. 12 illustrates a fifth embodiment of an implantable valve 100. The valve 100 is similar to the valve 90, and therefore comprises a generally cylindrical body 102. However, the outlet 104 has a generally S-shaped cross-section that is formed by two opposed S-shaped leaflets 106 and 108. Because the S-shape, the outlet orifice 110 (shown in the closed state) is larger than the outlet orifices of the previously-described valves.

FIG. 13 illustrates a sixth embodiment of an implantable valve 120. The valve 120 is similar to the valve 90, and therefore comprises a generally cylindrical body 122. However, the outlet 124 comprises three leaflets 126 that together form the outlet orifice 128 (shown in the closed state).

FIG. 14 illustrates a seventh embodiment of an implantable valve 130. The valve 130 is similar to the valve 90, and therefore comprises a generally cylindrical body 132. However, the outlet 134 comprises four leaflets 136 that together form the outlet orifice 138 (shown in the closed state).

The valves of the present disclosure are designed to accommodate the anatomy and mechanical properties of veins. The valves therefore can have elasticity for proper valve function. When provided, the valve leaflets in particular can have sufficient elasticity to flex from a substantially open position to a closed position with the application of reversing flow and pressure. In some embodiments, the leaflets deform under bending forces with a bending stiffness or modulus of elasticity of the leaflets is less than 5 MPa, preferably between 0.1 to 4 MPa, or more preferably less than 1 MPa. The valve leaflets may be more compliant than the valve body. Preferably, the valve body will be stiffer to maintain an open shape, while the leaflets may have less stiffness. Thus, the modulus of elasticity of the body of the valve is greater than 0.1 MPa and preferably greater than 0.5 MPa, or even more preferably greater than 1 MPa. The stiffness can be created by increasing the thickness of the body or by using a stiffer material. Both modifications in thickness and material to alter the stiffness are included in the present disclosure.

The leaflets are fatigue resistant, allowing for many cycles of leaflet bending. The disclosed valves are distinguished in their ability to withstand many cycles of bending without a hinge. In some embodiments, the valve leaflets are made of a hydrophilic synthetic polymer with a large opening for antegrade flow. The ability to withstand closure with 300 mm Hg back pressure for hundreds of thousands of cycles depends on the specific shape and strength of the juncture at the base of the valve leaflets to the valve body.

In some embodiments, the valves have the ability to expand elastically, in the radial direction, axial direction, or both. The entire valve may be elastically expandable, or certain portions of the valve may be elastically expandable to various degrees. For instance, in some embodiments, the valve can elastically expand in the radial direction and increase its radius by a value of about 0 R to about 0.5 R, preferably by at least about 0.2 R, where R is the inner radius of the tube. During such expansion, the valve does not tear or break and experiences negligible plastic deformation. Embodiments of the valve can elastically expand in the radial direction, increasing in radius by a value of about 0 R to about 1.0 R, preferably by about 0.5 R, where R is the inner radius of the central portion of the tube. During such expansion, the valve does not tear or break and experiences negligible plastic deformation.

In some embodiments, the valves can also elastically expand in length by a value of about 0 L to about 0.5 L, preferably by at least about 0.3 L, while experiencing negligible plastic deformation, without tearing or breaking, where L is the total length of the valve in the longitudinal or axial direction.

Conversely, the disclosed valves have the ability to compress into a smaller space. For delivery into the vein using endoscopic techniques, it is desirable for the valve to compress into a small sheath for delivery. Preferably, the valves of the present disclosure can be compressed into a sheath with a 20 French diameter, preferably 16 French, and even more preferably a 12 French catheter size.

As noted above, the valves of the present disclosure can be fabricated using a single material that is cast or injected into a mold. This makes the production of the valves fairly simple and economic, and the benefits of the financial and temporal savings can be passed along to the patient and surgeon. Preferably, the material used to make the valves is biocompatible and has low thrombogenicity. Suitable materials include, but are not limited to, polyurethanes, polyesters, polyethylenes, hydrogels, silastics, collagens, elastins, room temperature vulcanized (RTV) rubbers, and silicones. A second material in a particulate form such as, but not limited to, fibers, filaments, and/or grains can be added into the valve body to create a composite material, altering the stiffness and improving the fatigue life of the valve. This aspect of the design is advantageous in that it gives the manufacturer and the surgeon the ability to tailor the valves to a patient's specific clinical needs.

The valves of the present disclosure can be implanted into a patient through several modes known to those of skill in the art. To minimize trauma, pain, and potential for infection, the valves can be delivered to the implantation site via an intravenous catheter. The flexibility and durability of the valves make them highly deliverable via a catheter. The valves can then be fixed into position using a fixation device, such as, but not limited to, a balloon-expandable stent, a self-expanding stent, hooks or barbs, or other endovascular implantation techniques known to those in the field.

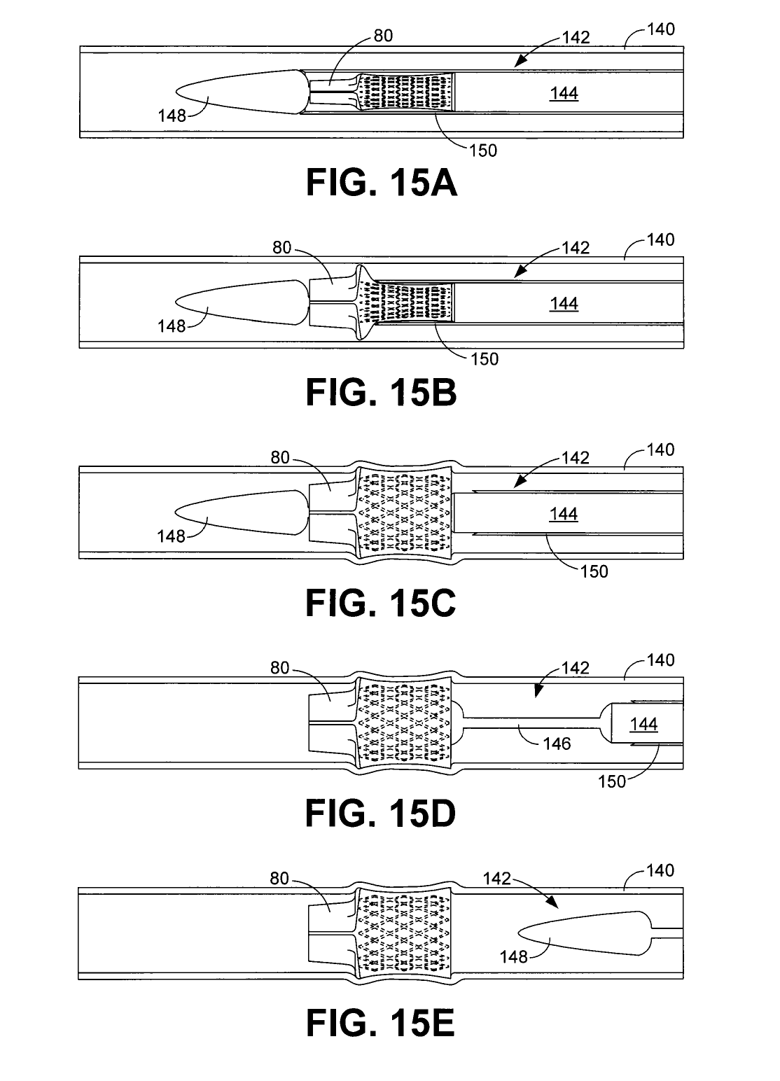

An exemplary mode of implantation and fixation involves delivery via an endovascular insertion. This involves first delivering valve of the present disclosure to the implantation site via catheter, and then securing it inside the vessel using suitable fixation techniques such as stents, barbs, sutures, vascular ingrowth, or combinations thereof. FIGS. 15A-15E illustrate an embodiment of a method for positioning a valve (valve 80 in this example) inside a vein 140. The valve 80 is delivered to the inside of the vein 140 using a delivery tool 142. In the example embodiment of FIGS. 15A-15E, the delivery tool 142 generally comprises a shaft 144, a narrow neck 146 (see FIG. 15D), and a tapered head 148. The valve 80 is disposed about the neck 146 in a compressed state and is maintained in the compressed state by a retractable sheath 150 that surrounds the valve.

As is shown in FIG. 15A, the delivery tool 142 is passed through the vein 140 until the valve 80 is positioned at a desired implantation site within the vein. Referring next to FIG. 15B, the retractable sheath 150 is retracted while maintaining the delivery tool 142 and the valve 80 in position along the length of the vein 140. As indicated in FIG. 15B, retraction of the sheath 150 enables the compressed valve 80 to open or expand within the vein 140. In FIG. 15C, the sheath 150 has been fully withdrawn from the valve 80 so that the valve 80 has fully expanded. Such expansion is in part due to the body of the valve 80 resuming its natural, uncompressed shape. In cases in which the valve 80 comprises a self-expanding stent, the self-expansion of the stent further aids in expansion of the valve. As is apparent in FIG. 15C, the valve 80 can exert force against the walls of the vein 140 when the valve is in the fully-expanded state to help hold the valve in place.

Once the valve 80 has fully expanded, the delivery tool 142 can be withdrawn, as is depicted in FIGS. 15D and 15E. As described above, the valve 80 can, optionally, be fixed in place using a suitable fixation means, such as sutures.

Delivery can also be accomplished by performing a venotomy, which involves making a longitudinal incision through the wall of the vessel. The incision should be long enough to stretch open the vein wall and insert the valve by hand. Generally, non-absorbable sutures are used for venous surgery, and are comprised of materials such as, but not limited to, silk or polypropylene. In particular regard to vascular surgery, suture size preferably ranges from about 5-0 to about 8-0. Interrupted sutures will give the greatest knot security, and can be placed in a longitudinal or circumferential direction, through the inlet and outlet of the valve. The number of sutures needed per valve will vary due to the diameter of the valve.

When using any mode of delivery, preferably the valve is positioned such that the plane in which the leaflets make contact is tangent to the circumferential direction of the limb. This allows the valve to perform appropriately even when compressed by the deep fascial muscular pressure. The shape of the valve leaflets is important to create this physiologic behavior.

The fixation of the valve to the vein wall can also be achieved by the incorporation of fibers to induce a biological tissue response after placement. This incorporation of an inflammatory agent such as polyester or polyethylene into the external wall of the valve body is preferred. It may be advantageous to facilitate intimal growth and healing of the vessel to improve circumferential sealing of the valve. This can be accomplished by incorporating a woven, knitted, or otherwise porous sheath of biocompatible material onto the outer surface of the valve body. Suitable materials include, but are not limited to, polyethylene terephthalate (PET), expanded polytetrafluoroethylene (ePTFE), or a similar material that has been shown to facilitate intimal growth in vascular graft applications.

The leaflets of the valves are made with a synthetic material that has elastic strength and low thrombogenicity. Many synthetic materials do not have strength and low thrombogenicity. Desirably, the vein valves of the present disclosure have both strength to withstand 300 mm Hg back pressure and have lower thrombogenicity than a similar valve made with a cardiac polyester in an in vitro system of blood flow. Platelet attachment to the valves is very low compared to other non-native valves. Appropriate materials for the valve and valve leaflets of the present disclosure are discussed further below.

In certain embodiments, anti-thrombogenic or thrombolytic agents including, but not limited to, heparin, sodium warfarin, calcium, or albumin are incorporated into the valves to help improve the response of the surrounding tissue and fluid to the introduction of a prosthetic valve. In such embodiments the agents may be incorporated on the surface of the valves of and/or into the valve material, and can be released actively or passively, at varying rates.

In yet other embodiments, a radiopaque material is incorporated with the valves to allow a clinician to track the motion and position of the valves during catheter delivery via fluoroscopy. This is advantageous because the valve's performance will be optimized if placed in the correct location, and this method allows the clinician to accurately know the valve's location inside the body at any given time during the implantation procedure.

Embodiments of the present disclosure entail designing the valves based on venous anatomy, physiology, and local biomechanics. Embodiments also entail fabricating the valves in a manner that is economical, timely, tailored to allow appropriate quality control measures, makes use of readily available materials, and allows customizing the design for specific clinical needs. The valves can be produced using low-cost casting methods allowing for an economic product that can be made with good manufacturing practices and sterilized in accordance with USFDA guidelines.

The shape and size of the valves can impact the efficacy of the valve. Preferably, the valves are sized relative to the vessel that it will be implanted in. When implanting the valves into human deep veins, the outer diameter of the body of the valves may range from about 0.75 D to about 1.50 D, where D is the un-collapsed inner diameter of the vein at low pressures. Preferably, the body outer diameter may be from about 0.9 D to about 1.3 D, and more preferably, from about 1.0 D to about 1.2 D. In certain embodiments the outer diameter of the body may be from about 1 millimeter to about 50 millimeters.

The length of the valves may be from about 0.5 D to about 4 D, more preferably from about 1 D to about 4 D, and most preferably about 2 D to about 3 D. In certain embodiments, the length of the valves may be from about 2 millimeters to about 50 millimeters. The thickness of the valve walls (e.g., the walls of the valve body) may be from about 0.01 D to about 0.2 D, and most preferably about 0.05 D to about 0.15 D.

The thickness of the leaflets may be from about 0.01 D to about 0.2 D, and preferably may be from about 0.05 D to about 0.15 D. Alternatively, the valve leaflets may have a thickness of less than 1 mm, preferably less than 0.5 mm. The thickness will allow a larger opening between the leaflets and permit free flow of blood. In some embodiments, the valves have two leaflets to keep the valve relatively simple to manufacture and make the valves more robust.

It is noted that the leaflet shape in some embodiments of the valve of the present device is non-anatomic. Natural vein valve leaflets are attached directly to the vein wall and are shaped like parabolas or “U”s.

The valves of the present disclosure are preferably made of a synthetic organic polymer that is hydrophilic and biocompatible. The incorporation of an organic hydrophilic material renders the valve less thrombogenic and less immunogenic. In some embodiments, the valves are made of a single material to improve the control of quality, ease of manufacture, and cost of fabrication. Preferably, the valves are made primarily of a synthetic material. More preferably, the material used is also flexible, durable, and commercially available or easy to make. Suitable materials for use in creating the valves of the present disclosure include, but are not limited to, polyurethanes, polyesters, polyethylenes, hydrogels, collagen, elastin, and silicone. One preferred material for use comes from the hydrogel group: poly(vinyl alcohol) cryogel (PVA cryogel), which is disclosed in U.S. Pat. No. 5,981,826, which is hereby incorporated by reference into the present disclosure. PVA cryogel is a hydrogel that has been shown to have low thrombogenicity (Miyake H, Handa H, Yonekawa Y, Taki W, Naruo Y, Yamagata S, Ikada Y, Iwata H, Suzuki M, New Small-Caliber Antithrombotic Vascular Prosthesis: Experimental Study, Microsurgery. 1984;5(3):144-50).

PVA cryogel can be manufactured as described in U.S. Pat. No. 5,981,826. In using any of the aforementioned prescribed materials, molding is a preferred method to fabricate the valve of the present disclosure, and can be conducted by those familiar with the general art of molding.

Preferably, the valve contains a radiopaque marker to facilitate delivery, orientation, and placement of the valve using intravenous catheter approaches. Such markers are preferably biocompatible, have low thrombogenicity, and are preferably cast into the valve inlet and outlet. Radiopaque marker(s) can be added to the valve via methods commonly known to those familiar with the art of manufacturing medical devices. Exemplary radiopaque markers suitable for use with a valve according to the present invention include, but are not limited to, platinum, iridium, and nickel titanium alloys.

The descriptions above detailing certain exemplary embodiments contain specificities and are intended only to best illustrate the design and function of the valve of the present disclosure for a person of ordinary skill in the art to become knowledgeable and enabled to utilize the present disclosure for its appropriate purposes. The descriptions are neither exhaustive nor meant to limit the scope of the present disclosure to the specificities disclosed above. Many variations and modifications may be made to the above-described embodiments of the present disclosure without departing substantially from the spirit and principles of the present disclosure. All such modifications and variations are intended to be included herein within the scope of this disclosure and protected by the following claims.

Having generally described prosthetic valves according to the present disclosure and methods of making and using such valves, the examples that follow describe some specific embodiments. While embodiments of the valves and methods of making and using the valves are described in connection with the following examples and the corresponding text, there is no intent to limit embodiments to these examples. On the contrary, the intent is to cover all alternatives, modifications, and equivalents included within the scope of the disclosure.

1. An implantable prosthetic valve comprising:
a body that defines an inlet end and an inlet orifice, the body being constructed of a flexible biocompatible material;
an outlet that extends from the body and that defines an outlet end and an outlet orifice, the outlet being constructed of a flexible biocompatible material; and
an inner passage that extends through the body and the outlet from the inlet orifice to the outlet orifice to enable fluid to flow through the valve;
wherein the outlet orifice is open when the valve is in its natural unloaded state and is closed when fluid pressure is applied to the outlet from a position downstream of the outlet.

2. The valve of claim 1, wherein the body and the outlet are made of a biocompatible polymeric material.

3. The valve of claim 1, wherein the body and the outlet are made of a biocompatible hydrogel.

4. The valve of claim 1, wherein the body and the outlet are unitarily formed from a biocompatible hydrogel.

5. The valve of claim 1, wherein the body is generally cylindrical.

6. The valve of claim 5, wherein outer surfaces of the body curve inwardly from the inlet end toward the outlet.

7. The valve of claim 1, wherein the outlet is generally cylindrical.

8. The valve of claim 1, wherein the outlet has a smaller cross-section than the body.

9. The valve of claim 8, further comprising a ledge formed at a junction of the body and the outlet.

10. The valve of claim 9, wherein the ledge has a generally planar surface substantially perpendicular to a longitudinal direction of the valve and a curved outer edge.

11. The valve of claim 1, wherein the outlet comprises opposed flexible leaflets that collapse against each other when the outlet closes.

12. The valve of claim 11, wherein the outlet comprises two flexible leaflets that are joined at opposed seams.

13. The valve of claim 12, wherein the outlet and the outlet orifice have a generally lemon-shaped cross-section.

14. The valve of claim 1, further comprising longitudinal ribs that are provided on the outer surfaces of the body.

15. The valve of claim 1, further comprising an integral stent.

16. The valve of claim 15, wherein the stent is encapsulated within the valve.

17. The valve of claim 15, wherein the stent is a self-expanding metallic stent.

18. The valve of claim 1, wherein the outlet is frustoconical.

19. The valve of claim 1, wherein the outlet has an S-shaped cross-section.

20. An implantable prosthetic valve comprising:
a generally cylindrical body that defines an inlet end and an inlet orifice;
a generally cylindrical outlet that extends from the body and that defines an outlet end and an outlet orifice, the outlet having a smaller cross-section than the body; and
an inner passage that extends through the body and the outlet from the inlet orifice to the outlet orifice to enable fluid to flow through the valve;
wherein the body and the outlet are unitarily formed of a biocompatible hydrogel;
wherein the outlet orifice is open when the valve is in its natural unloaded state and is closed when fluid pressure is applied to the outlet from a position downstream of the outlet.

21. The valve of claim 20, further comprising a ledge formed at a junction of the body and the outlet.

22. The valve of claim 21, wherein the ledge has a generally planar surface substantially perpendicular to a longitudinal direction of the valve and a curved outer edge.

23. The valve of claim 20, wherein the outlet comprises two opposed flexible leaflets that collapse against each other when the outlet closes, the leaflets being joined at opposed seams.

24. The valve of claim 23, wherein the outlet and the outlet orifice have a generally lemon-shaped cross-section.

25. The valve of claim 20, further comprising an integral self-expanding metallic stent that is encapsulated within the valve.
